(19) United States
(12) Patent Application Publication
(10) Pub. No.: US 2025/0288774 A1
(43) Pub. Date: Sep. 18, 2025

(54) Urinary Catheter Devices and Methods

(71) Applicant: C. R. Bard, Inc., Franklin Lakes, NJ (US)

(72) Inventors: Pooja Bhuvanesh Kulkarni, Pune (IN); Sudev Geetha Surendran, Kozhikode District (IN); James David Hughett, Sr., Monroe, GA (US); Ronald N. Legaspi, Alpharetta, GA (US)

(21) Appl. No.: 18/859,513

(22) PCT Filed: Apr. 25, 2022
(86) PCT No.: PCT/US22/26177

§ 371 Date: Oct. 23, 2024

(51) Int. Cl. A61M 25/00; A61M 25/01

(52) U.S. Cl. CPC A61M 25/0017; A61M 25/002; A61M 25/0029; A61M 25/0136

(57) ABSTRACT

Disclosed herein is a urinary catheter generally configured for self-deployment. The catheter includes a closed volume within the lumen of the catheter containing a lubricant. The proximal end and the distal end of the catheter tube are coupled with a handling member disposed along the catheter tube to define a compact packaging shape of the catheter tube including one or more loops. The closed volume is breached by separating the proximal and distal ends away from the handling member in a single step via simultaneous application of separating forces to the proximal and distal ends. The handling member includes a pull ring for applying an oppositely directed separating force to the handling member. The catheter includes an inlet section configured of insertion into the patient and an inlet collar is slidably coupled with the inlet section. where in the inlet collar serves as a guide during insertion of the catheter.

### BACKGROUND

Urinary incontinence causes a person to lose control over their bladder resulting in loss of large or small quantities of urine. This can be caused due to multiple reasons. A paraplegic or tetraplegic user loses the control of their bladder and they need to empty their bladder using urinary catheters. These can be indwelling (the catheter is placed for a longer duration inside the bladder and attached to a urine bag to collect the urine) or they can be intermittent (the catheter is periodically inserted into the bladder and the urine is drained from it either directly in appropriate drainage area or inside a urine bag). Intermittent catheters are preferred in the cases where the user can self-catheterize or wishes to not be seen with a urine bag. The intermittent catheters allow the user a freedom of mobility.

A catheter can be pre-lubricated. In some cases, the lubricant can spill while opening the package which can soil or stain the user's clothing. In some cases, urinary catheters include a packaging shape that is not conducive to carry on one's person, such as within a pocket of clothing. Furthermore, in some cases, the catheters may be difficult to manipulate during self-catheterization.

Disclosed herein are urinary catheters and methods of catheterization that address the items described above.

### SUMMARY

Briefly summarized, disclosed herein is a urinary catheter that includes a catheter tube defining a distal end and a proximal end. The catheter tube includes an inlet section extending proximally away from the distal end, where the inlet section is configured for advancement within a urethra of a user. The catheter tube also includes an outlet section extending proximally away from the inlet section. A lumen of the catheter tube extends between the proximal end and the distal end. The proximal end of the catheter tube is coupled with an outlet connection member of the catheter to define a loop of the catheter tube.

In some embodiments, the distal end of the catheter tube is coupled with an inlet connection member of the catheter to define a closed volume extending along the lumen from the distal end to the proximal end.

In some embodiments, the distal end is configured to selectively couple with and decouple from the inlet connection member, and the proximal end is configured to selectively couple with and decouple from the outlet connection member.

In some embodiments, a lubricant is contained within the closed volume.

In some embodiments, the catheter further includes an inlet collar coupled with the catheter tube, where the inlet collar is positionable along the inlet section between the distal end of the catheter tube and a proximal end of the inlet section.

In some embodiments, the inlet collar includes the outlet connection member.

In some embodiments, the catheter further includes a handling member attached to the catheter tube along the outlet section, where the handling member is configured for grasping by a user during deployment of the catheter.

In some embodiments, the handling member includes grasping features disposed along an exterior surface of the handling member to enhance grasping of handling member by the user, and the grasping features include one or more of protrusions, depressions, axial ribs, axial troughs, annular ribs, annular troughs, or a textured surface.

In some embodiments, the handling member includes the outlet connection member and the inlet connection member so that the outlet section defines a loop and the inlet section defines a loop.

In some embodiments, the proximal end is configured to decouple from the outlet connection member in response to an axial separation force applied to the outlet section, and the distal end is configured to decouple from the inlet connection member in response to an axial separation force applied to the inlet section.

In some embodiments, the distal end is coupled with the inlet connection member via the inlet collar.

In some embodiments, the outlet connection member and the inlet connection member are arranged to define a parallel orientation of the proximal and distal ends of the catheter tube.

In some embodiments, the outlet connection member and the inlet connection member are arranged side by side to enable simultaneous application of the axial forces to the outlet section and the inlet section.

In some embodiments, the catheter further includes an outlet collar fixedly attached to catheter tube adjacent the proximal end, and the proximal end of the catheter tube is coupled with the outlet connection member via the outlet collar.

In some embodiments, the inlet collar and/or the outlet collar include one or more of the grasping features disposed along an outside surface.

In some embodiments, the inlet section includes a number of openings adjacent the distal end, where the openings extend between the lumen and an outside surface of the inlet section, and where the inlet collar is configured to cover the openings when the inlet collar is positioned at the distal end.

Also disclosed herein is a urinary catheter that includes a catheter tube configured for insertion within a urethra, where the catheter tube defines a lumen extending between a proximal end of the catheter tube and a distal end of the catheter tube. The catheter is transitionable between a packaging state and a ready-to-use state such that: (i) in the packaging state, a lubricant is sealably contained within the lumen between the proximal end and the distal end, and (ii) in the ready-to-use state, the lubricant is drained from the lumen.

In some embodiments, when the catheter is disposed in the packaging state, the catheter tube defines a shape having two or more loops. In further embodiments, when the catheter is disposed in the packaging state, the proximal end is positioned adjacent the distal end. In additional embodiments, when the catheter is disposed in the packaging state, the proximal end and the distal end are oriented parallel with each other. In still further embodiments, when the catheter is disposed in the packaging state, the proximal end and the distal end face the same direction.

Also disclosed herein is a method for draining urine from a patient's bladder. The method includes: (i) providing a catheter having a catheter tube configured for insertion into the patient bladder; (ii) breaching a closed volume of the catheter to release a lubricant from the closed volume, where the closed volume is defined by a lumen of the catheter tube between a proximal end and a distal end; and (iii) inserting the catheter tube into the patient bladder to establish a flow of urine through the lumen.

In some embodiments, breaching the closed volume includes breaking a first seal at the proximal end, and breaking a second seal at the distal end.

In some embodiments, breaching the closed volume includes separating the proximal end away from the distal end.

In some embodiments, the method further includes slidably displacing an inlet collar of the catheter proximally along the catheter tube away from the distal end to expose a distal tip of the catheter tube.

In some embodiments, inserting the catheter tube includes grasping the inlet collar to guide the distal tip of the catheter tube into a urethra of the patient.

In some embodiments, the catheter includes a handling member attached to the catheter tube, and breaching the closed volume includes applying a pulling force to a pull ring of the handling member.

In some embodiments, inserting the catheter tube includes grasping the handling member to apply an axial force to the catheter tube.

In some embodiments, breaching the closed volume includes displacing the proximal end and the distal end away from the handling member.

In some embodiments, the method further includes: (i) withdrawing the catheter tube from the patient bladder, and (ii) coupling both the distal end and the proximal end to the handling member to re-establish the closed volume.

These and other features of the concepts provided herein will become more apparent to those of skill in the art in view of the accompanying drawings and following description, which disclose particular embodiments of such concepts in greater detail.

### BRIEF DESCRIPTION OF THE DRAWINGS

Embodiments of the disclosure are illustrated by way of example and not by way of limitation in the figures of the accompanying drawings, in which like references indicate similar elements and in which:

FIG. 1A is an illustration of a urinary catheter in a packaging state, in accordance with some embodiments;

FIG. 1B is an illustration of the urinary catheter of FIG. 1A in an unpackaged state, in accordance with some embodiments;

FIG. 1C is a detailed illustration of a distal end portion of the urinary catheter of FIG. 1A, in accordance with some embodiments;

FIG. 1D is an illustration of the urinary catheter of FIG. 1A in a draining state, in accordance with some embodiments;

FIG. 1E is an illustration of the urinary catheter of FIG. 1A in a ready-to-use state, in accordance with some embodiments;

FIG. 1F is an illustration of the urinary catheter of FIG. 1A inserted within a patient, in accordance with some embodiments;

FIG. 2A is an illustration of a second embodiment of a urinary catheter in a packaging state, in accordance with some embodiments;

FIG. 2B is an illustration of the urinary catheter of FIG. 2A in a partially unpackaged state, in accordance with some embodiments;

FIG. 2C is an illustration of the urinary catheter of FIG. 2A in a fully unpackaged state, in accordance with some embodiments;

FIG. 3A is detailed illustration of a first embodiment of the handling member of the catheter of FIGS. 2A-2B, in accordance with some embodiments; and

FIG. 3B is detailed illustration of a second embodiment of the handling member of the catheter of FIGS. 2A-2B, in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

The phrases “connected to,” “coupled with,” and “in communication with” refer to any form of interaction between two or more entities, including but not limited to mechanical, and fluid interaction. Two components may be coupled with each other even though they are not in direct contact with each other. For example, two components may be coupled to each other through an intermediate component.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations may be made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially straight” is recited with respect to a feature, it is understood that in further embodiments, the feature can have a precisely straight configuration.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

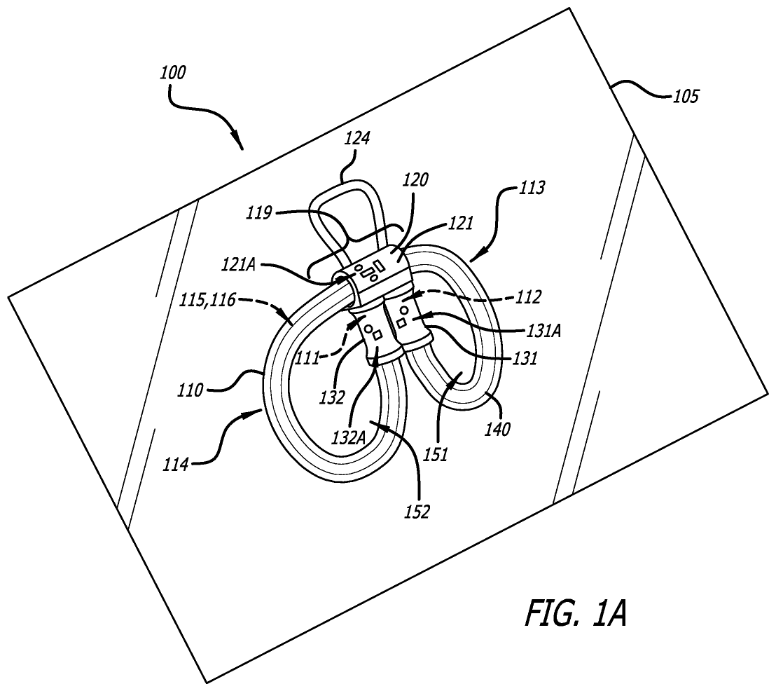

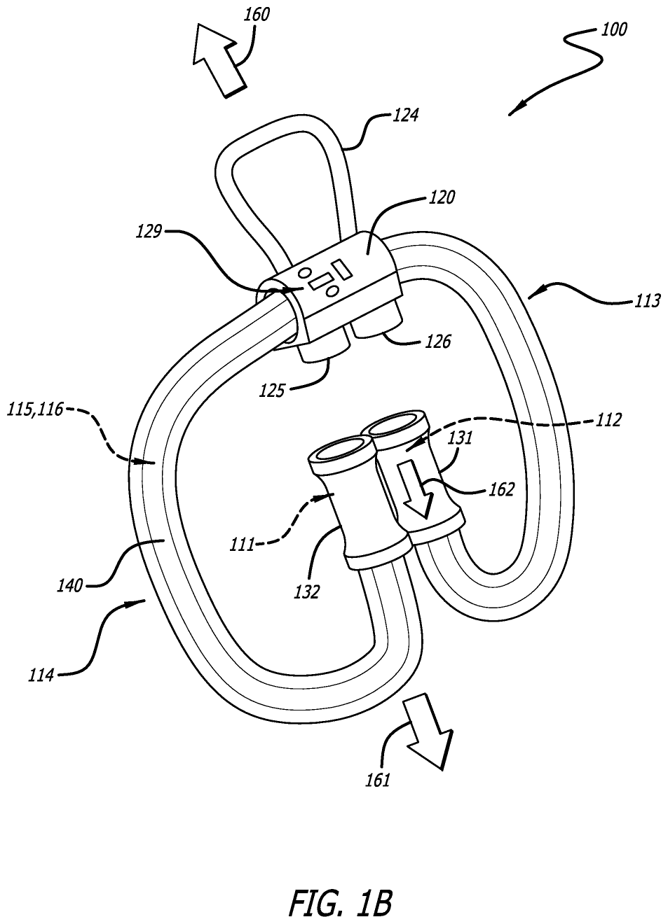

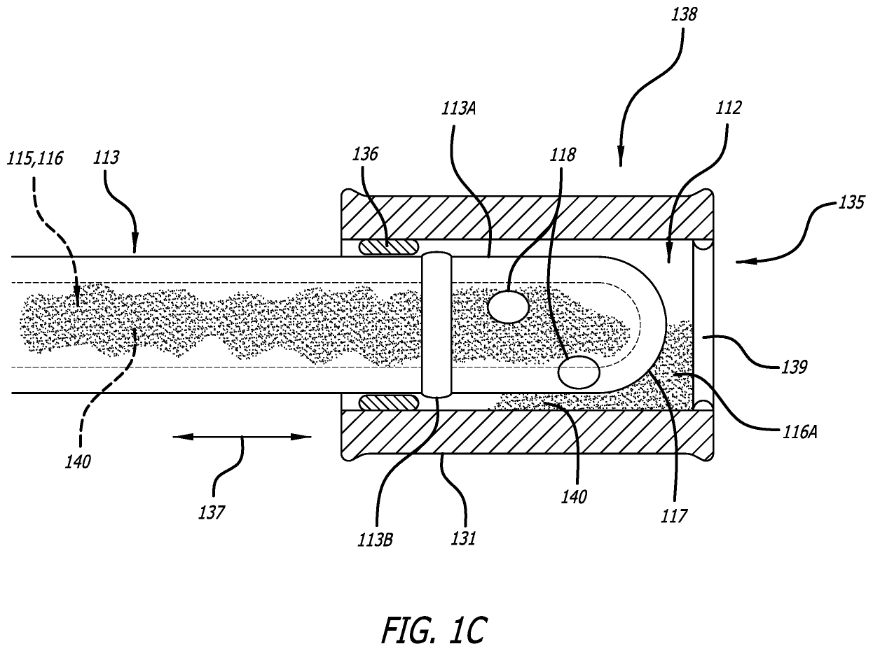

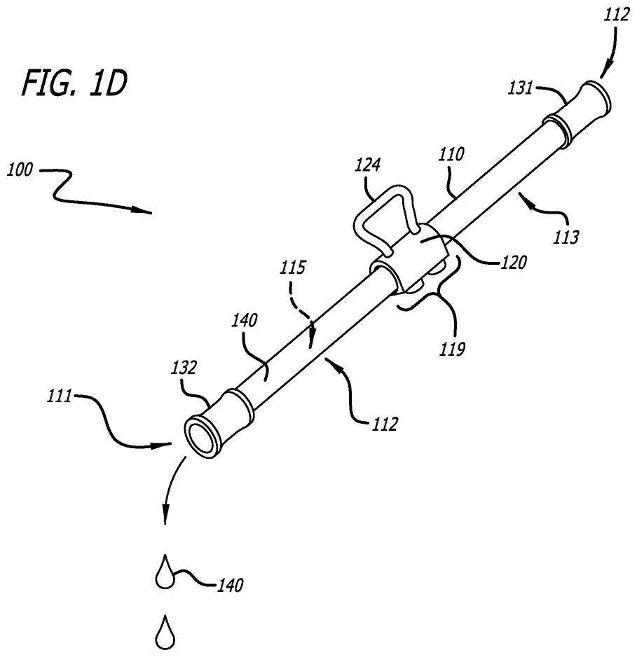

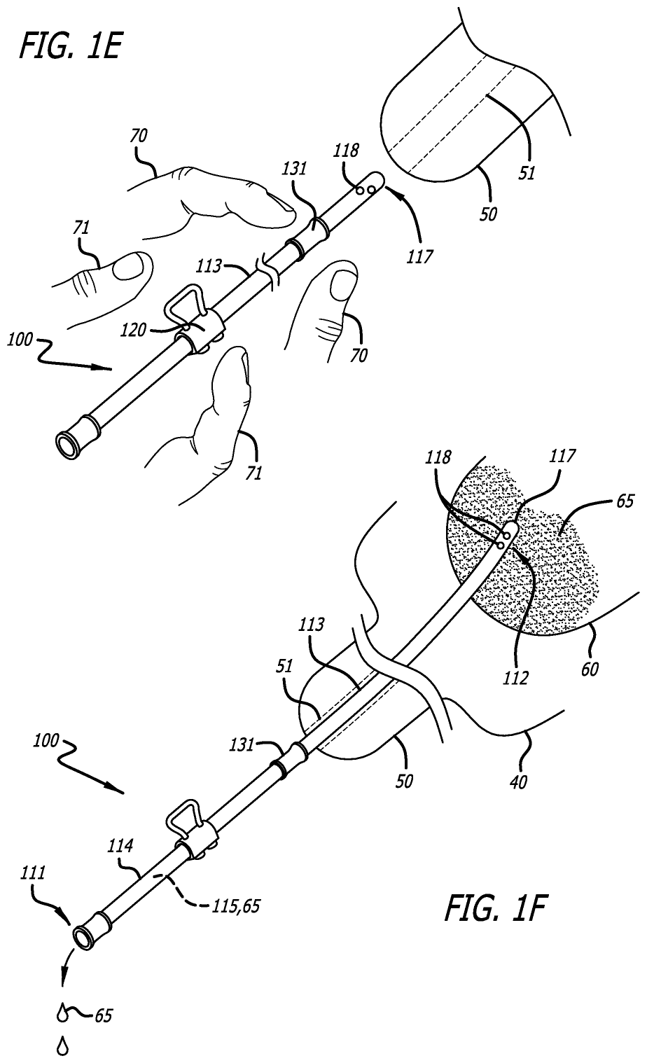

FIGS. 1A-IF illustrate a first embodiment of a urinary catheter in various states of use. The urinary catheter (catheter) 100 is generally configured for self-deployment by a user (also referred to herein as a patient) to drain the user's bladder of urine. The catheter 100 as illustrated is generally configured for use by a male patient. However, the catheter 100 may be configured for use by a female patient without departing from the features and functional aspects of the catheter 100 as shown and described below. The catheter 100 may be used by the patient in various settings, such as at a medical facility, at a home, or at a public restroom, for example.

The catheter 100 is generally configured to be used by the patient multiple times during a day. More specifically, the patient may deploy multiple catheters 100 (one at a time) during the day. As such, the patient my keep and carry one or more catheters 100 on his person, such as in a pocket of clothing or a purse, for example. As such, the catheter 100 may be disposed in packaging state having a compact shape consistent with placement of the catheter 100 within the pocket of clothing or purse. The catheter 100 is also configured to maintain a sterility of the catheter 100 in the packaging state.

In some instances, the user may be concerned about soiling clothing during the urine draining process, via the urine or a lubricant of the catheter, for example. Hence, the catheter 100 is configured for deployment while also inhibiting the spilling of a lubricant of the catheter 100 on clothing or other items during use. Furthermore, the catheter 100 is generally configured to contain any residual urine within the catheter tubing after use.

As the catheter 100 is configured for self-deployment, the catheter 100 may be configured for ease in handling (i.e., gripping, orienting, guiding, or applying a force, etc.) by a user during deployment. As such, the catheter 100 may be configured for deployment by users having limited dexterity.

FIG. 1A illustrates a perspective front view of the catheter 100 in a packaging state. The catheter 100 may be disposed in the packaging before use. The catheter 100 may be disposed in an outer package 105, such as a bag or pouch, for example. The catheter 100 generally includes a catheter tube 110 having a proximal end 111 and a distal end 112. The catheter tube 110 may be formed of a material consistent with defining a soft material sheath. A lumen 115 extends along the catheter tube 110, where the lumen 115 is sized to facilitate urine flow therethrough from the distal end 112 to the proximal end 111 during use. The catheter tube 110 includes an inlet section 113 and an outlet section 114. The inlet section 113 is configured for insertion into and advancement along a urethra of the user to the patient's bladder. As such, the inlet section 113 defines a length sufficient to extend from the end of a penis to the bladder of the patient. Similarly, the inlet section defines an outside diameter compatible with placement within the urethra. The outlet section 114 is configured for use outside of the patient body and defines a length compatible with placement of the proximal end over a urine receptacle, such as a toilet, for example, during use.

The catheter 100 includes a handling member 120 configured to provide an insertion aid to the user. A handling member 120 is disposed along a medial section 119 of the catheter tube 110, where the medial section 119 is disposed along a proximal portion of the outlet section 114. The handling member 120 is configured for gripping during use by the patient. The handling member 120 includes an outside surface 121. Gripping features 121A are disposed on the outside surface 121 to enhance a grip-ability of the handling member 120. The gripping features 121A may include protrusions, depressions, ribs, troughs, a rough surface, or any combination thereof. The handling member also includes a pull ring 124 as further described below. The handling member 120 may be attached to the catheter tube 110 via any suitable method, such as adhesive bonding, heat bonding, insert molding, or an interference fit, for example.

The catheter tube 110 includes an inlet collar 131 coupled with the inlet section 113 to also provide an insertion aid to the user. The inlet collar 131 is configured for gripping by the user during use. As such, an outer surface of the inlet collar 131 may include gripping features 131A, such as the gripping features 121A.

The catheter tube 110 may also include an outlet collar 132. The outlet collar 132 is fixedly attached to the catheter tube 110 at the proximal end 111. In some embodiments, the outlet connector may include gripping features 132A, such as the gripping features 121A. In some embodiments, the outlet collar 132 may be omitted.

The proximal end 111 and the distal end 112 are coupled with handling member 120 in the packaging state. When the proximal end 111 and the distal end 112 are coupled to the handling member 120, the handling member 120 sealably closes the proximal end 111 and the distal end 112 to define a closed volume 116 within the lumen 115.

The coupling of proximal end 111 and the distal end 112 with handling member 120 defines a packaging shape of the catheter 100 in the packaging state. In some embodiments, the packaging shape may include a number of loops such as the loop 151 of the inlet section 113 and the loop 152 of the outlet section 114. In some embodiments, the proximal end 111 and the distal end 112 may be position laterally adjacent one another in a parallel fashion as shown so that the proximal end 111 and the distal end 112 face the same direction. The proximal end 111 and the distal end 112 may be coupled with handling member 120 on a side opposite the pull ring 124.

The catheter 100 includes a lubricant 140 disposed within the closed volume 116 in the packaging state. The lubricant 140 is configured to enable sliding displacement of the inlet section 113 along the urethra during use.

FIG. 1B illustrates the catheter 100 in an unpackaged state, i.e., a state where the proximal end 111 and the distal end 112 are decoupled from the handling member 120 such that the closed volume 116 is breached.

The handling member 120 includes an outlet coupling member 125 to facilitate coupling of the proximal end 111 to the handling member 120. The outlet coupling member 125 enables the proximal end 111 to be sealably coupled with the handling member 120. In some embodiments, the proximal end 111 is coupled with outlet coupling member 125 via a snap fit or detent. As such, the proximal end 111 be selectively coupled with and decoupled from the outlet coupling member 125. The proximal end 111 may be initially coupled with the outlet coupling member 125 at a manufacturing facility of the catheter 100.

In some embodiments, the coupling between the proximal end 111 and the outlet coupling member 125 may facilitate decoupling of the proximal end 111 from the outlet coupling member 125 via a separating force as applied by the user. More specifically, the user may apply a pulling force 160 to the pull ring 124 and opposite pulling force 161 to the outlet section 114 or the outlet collar 132 to separate the proximal end 111 from the outlet coupling member 125, e.g., the user apply a pulling force 160 and opposite pulling force 161 to overcome the snap fit.

The outlet coupling member 125 may also be configured to enable recoupling of the proximal end 111 to the handling member 120 so that the outlet coupling member 125 reseals (i.e., sealably closes off) the proximal end 111. More specifically, the outlet coupling member 125 may be configured to enable the user to recouple the proximal end 111 with the handling member 120 by applying a pushing force (i.e., opposite the pulling force 160) to the handling member 120 and an opposite pushing force (i.e., opposite the pulling force 161) to the outlet section 114 to reattach the proximal end 111 to the handling member 120.

Similarly, the handling member 120 includes an inlet coupling member 126 to facilitate coupling of the distal end 112 to the handling member 120. In some embodiments, the inlet coupling member 126 enables the inlet collar 131 to be coupled with the handling member 120. In some embodiments, the inlet collar 131 is coupled with inlet coupling member 126 via a snap fit. As such, the distal end 112 (via the inlet collar 131) be selectively coupled with and decoupled from the inlet coupling member 126. The inlet collar 131 may be initially coupled with the inlet coupling member 126 at the manufacturing facility of the catheter 100.

In some embodiments, the coupling between the distal end 112 and the inlet coupling member 126 may facilitate decoupling of the distal end 112 from the inlet coupling member 126 via a separating force as applied by the user. More specifically, the user may apply a pulling force 160 to the pull ring 124 and an opposite pulling force 162 to the inlet collar 131 to separate the inlet collar 131 from the inlet coupling member 126, e.g., the user apply the pulling force 160 and the opposite pulling force 162 to overcome the snap fit. In some embodiments, the opposite pulling force 162 may be applied to the inlet section 113.

Similarly, the inlet coupling member 126 may also be configured to enable recoupling of the inlet collar 131 to the handling member 120 so that the inlet coupling member 126 in combination with the inlet collar 131 reseals (i.e., sealably closes off) the distal end 112. More specifically, the inlet coupling member 126 may be configured to enable the user to recouple the distal end 112 with the handling member 120 by applying a pushing force (i.e., opposite the pulling force 160) to the handling member 120 and an opposite pushing force (i.e., opposite the pulling force 162) to the inlet collar 131 to reattach the distal end 112 to the handling member 120. Recoupling the distal end 112 with the handling member 120 combined with recoupling the proximal end 111 with the handling member 120 may redefine the closed volume 116 to contain any residual urine within the lumen after use.

During use, the user may orient the catheter 100 such that the pull ring 124 is positioned vertically above the proximal end 111 and the distal end 112 so that upon separation of the proximal end 111 and the inlet collar 131 from the handling member 120, the lubricant 140 is prevented from exiting the lumen 115 via proximal end 111 and/or the distal end 112. During use, the user may apply the opposite pulling forces 161, 162 at the same time to simultaneously separate the proximal end 111 and the inlet collar 131 from the handling member 120 in a single step.

FIG. 1C is a detailed illustration of a distal end portion of the urinary catheter 100, in accordance with some embodiments. The inlet collar 131 is shown in a packaging position 138 adjacent the distal end 112 of the catheter tube 110 consistent with the catheter 100 disposed in the packaging state. The inlet collar 131 is slidably coupled with the inlet section 113 as indicated by the double-ended arrow 137 so that the inlet collar 131 is positionable along the inlet section 113.

The inlet section 113 includes a distal tip 117 at the distal end 122 of the catheter tube 110. The distal tip 117 may include dome shape (e.g., a hemispherical shape) to facilitate insertion of the inlet section 113 into the urethra and further into the bladder of the user.

The inlet section 113 includes a number (e.g., 1, 2, 3, or more) of openings 118 extending between the lumen 115 and an outside surface 113A of the inlet section 113 adjacent the distal end 112. The openings 118 define a passageway for urine to flow into the lumen 115 from the bladder.

In some embodiments, the inlet collar 131 may be generally configured to define a fluid seal with the outside surface 113A of the inlet section 113 when the inlet collar 131 is disposed in the packaging position 138. In some embodiments, the inlet collar 131 may also define the fluid seal when the inlet collar 131 is displaced away from packaging position 138. The inlet collar 131 may include a sealing member 136 to define the fluid seal. The sealing member 136 may be defined by an inward protruding annual rib of the inlet collar 131 or a separate sealing device, such as an O-ring, for example. In some embodiments, the fluid seal 136 may be located adjacent a proximal end of the inlet collar 131.

In some embodiments, the inlet collar 131 may be constrained from distal displacement beyond the distal end 122, e.g., distally beyond the packaging position 138. In some embodiments, the inlet section 113 may include a radially expanded portion 113B, such as an annular protruding rib, for example. In such embodiments, the radially expanded portion 113B may be configured to engage the sealing member 136 so as to limit distal displacement of the inlet collar 131 along the inlet section 113 beyond the packaging position 138.

As discussed above, the inlet collar 131 is configured to define a fluid seal with the inlet coupling member 126 (see FIG. 1B). In some embodiments, the inlet collar 131 may include a sealing feature 139 at the open end 135 of the inlet collar 131, where the sealing feature 139 is configured to define the fluid seal between the inlet collar 131 and the inlet coupling member 126.

When the inlet collar 131 is coupled with the inlet coupling member 126, a closed compartment 116A may be optionally defined as an extension of the closed volume 116. As such, the closed compartment 116A is in fluid communication with the closed volume 116 via the openings 118. As the lubricant 140 is contained within the closed volume 116, the lubricant 140 may occupy a portion or more of the closed compartment 116A. As shown, the lubricant 140 within the closed compartment 116A may contact the outside surface 113A of the inlet section 113 thereby lubricating the outside surface 113A in preparation for advancement of the inlet section 113 along the urethra.

FIG. 1D illustrates the catheter 100 in a draining state, i.e., a state wherein the closed volume 116 is breached and the catheter 100 is oriented to enable the lubricant 140 to drain from the lumen 115. In other words, the user may de-coil/straighten the catheter tube 110 and orient the catheter tube 110 in a tilted position to allow gravity to cause lubricant to flow from the lumen 115. The catheter 100 may be tilled so that the distal end 112 is above the proximal end 111 or vice versa.

FIG. 1E illustrates the catheter 100 in a ready-to-use state, i.e., a state where the catheter 100 is ready for insertion into a urethra 51 of a penis 50. As discussed above the inlet collar 131 is slidably positionable along the inlet section 113. In the ready-to-use state, the inlet collar 131 is proximally positioned away from the distal end 112 to expose a distal tip 117.

The catheter 100 is generally configured for deployment (i.e., insertion into the bladder) by the user via grasping the inlet collar 131 with one hand 70 (e.g., between a thumb and finger of the one hand 70) and the grasping the handle member 120 with the other hand 71 (e.g., between a thumb and finger of the other hand 71). During deployment, the user may grasp the inlet collar 131 to position and/or orient the distal tip 117 with respect to the urethra 51. The user may grasp the handling member 120 to apply an axial force to the inlet section 113 to insert the inlet section 113 into the urethra 51. During insertion, the user may maintain a position of the inlet collar 131 with respect to the urethra 51 so that the inlet section 113 slides through the inlet collar 131 during advancement of the inlet section 113 toward the bladder.

FIG. 1F illustrates the catheter 100 in a fully deployed state, where the inlet section 113 is fully advanced along the urethra 51 so that the distal tip 117 is disposed within the bladder 60. The urine 65 is flowing into the openings 118, through the lumen 115, and out of the proximal end 111 of the outlet section 114. The inlet collar 131 is position sufficiently away from the distal end 112 toward the handling member 120 to expose a sufficient length of the inlet section 113 to extend from the open end of the urethra 51 to the bladder 60.

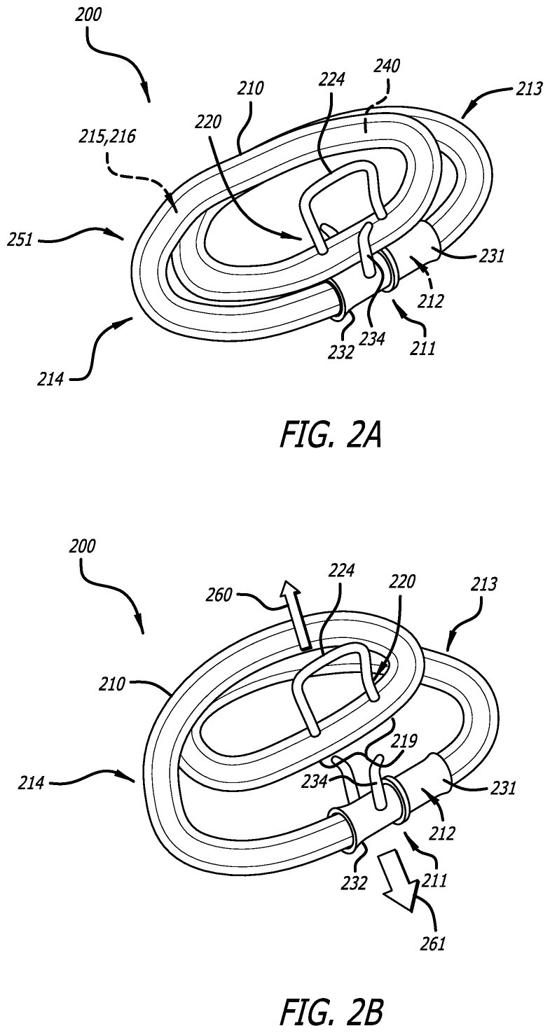

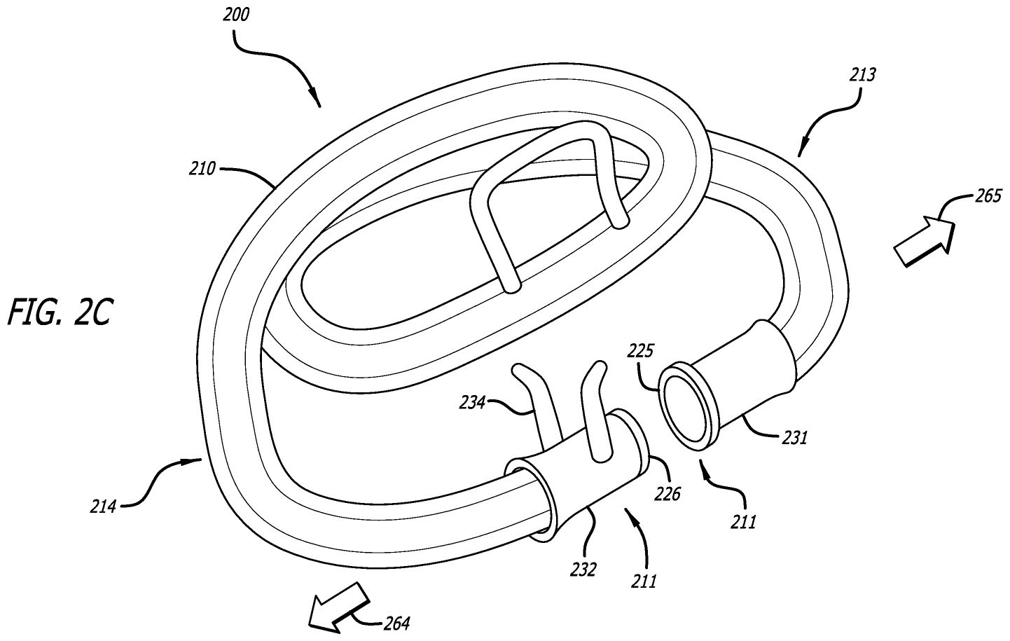

FIGS. 2A-2C illustrate another embodiment of a urinary catheter 200 that can, in certain respects, resemble components of the urinary catheter 100 described in connection with FIGS. 1A-1F. It will be appreciated that all the illustrated embodiments may have analogous features. Accordingly, like features are designated with like reference numerals, with the leading digits incremented to “2.” For instance, the catheter tube is designated as “110” in FIGS. 1A-1F, and an analogous catheter tube is designated as “210” in FIGS. 2A-2C. Relevant disclosure set forth above regarding similarly identified features thus may not be repeated hereafter. Moreover, specific features of the catheter 100 and related components shown in FIGS. 1A-1F may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the catheter 200. Any suitable combination of the features, and variations of the same, described with respect to the catheter 100 and components illustrated in FIGS. 1A-IF can be employed with the catheter 200 and components of FIGS. 2A-2C, and vice versa.

FIG. 2A illustrates the catheter 200 in a packaging state and FIG. 2B illustrates the catheter 200 is a partially unpackaged state. The urinary catheter (catheter) 200 is generally configured for self-deployment by the user to drain the user's bladder of urine. The catheter 200 is generally configured for use by a male user. However, catheter 200 may be configured for use by a female user without departing from the features and functional aspects of the catheter 200 as shown and described below. The catheter 200 may be used by the user in various settings, such as at a medical facility, at a home, or at a public restroom, for example.

The catheter 200 is generally configured to be used by the user multiple times during a day. More specifically, the user may deploy multiple catheters 200 (one at a time) during the day. As such, the user my keep and carry one or more catheters 200 on his person, such as in a pocket of clothing or a purser, for example. As such, the catheter 200 may disposed in packaging state having a shape consistent with placement of the catheter 200 within the pocket of clothing or purse.

As the catheter 200 is configured for self-deployment, the catheter 200 may be configured for ease in handling (i.e., gripping, orienting, guiding, or applying a force, etc.) by the user during deployment.

The catheter 200 may be disposed in the packaging state before use. Although not shown, the catheter 200 may be disposed in an outer package, such as the outer package 105, for example. The catheter 200 generally includes a catheter tube 210 having a proximal end 211 and a distal end 212. A lumen 215 extends along the catheter tube 210, where the lumen 215 is sized to facilitate urine flow therethrough from the distal end 212 to the proximal end 211 during use. The catheter tube 210 includes an inlet section 213 and an outlet section 214. The inlet section 213 is configured for insertion into and advancement along a urethra of the user to the user's bladder. As such, the inlet section 213 defines a length sufficient to extend from the end of a penis to the bladder of the user. Similarly, the inlet section defines an outside diameter compatible with placement within the urethra. The outlet section 214 is configured for use outside of the body and defines a length compatible with placement of the proximal end 211 over a urine receptacle, such as a toilet, for example during use.

A handling member 220 including a pull ring 224 is disposed along medial section 219 of the catheter tube 210 where the medial section 219 is disposed along a distal portion of the outlet section 214. The handling member 220 may be attached to the catheter tube 210 via any suitable method, such as adhesive bonding, heat bonding, insert molding, or an interference fit, for example.

The catheter tube 210 includes an inlet collar 231 coupled with the inlet section 213, where the inlet collar 231 is slidably positionable along the inlet section 213. The catheter tube 210 also includes an outlet collar 232 at the proximal end 211. In the packaging state, the outlet collar 232 is sealably coupled with the inlet collar 231 to define a closed space 216 within the lumen 215. A lubricant 240 is disposed with the closed space 216. The lubricant 240 is configured to enable sliding displacement of the inlet section 213 along the urethra during use.

The outlet collar 232 includes an attachment mechanism 234 configured to selectively secure the outlet collar 232 to the medial section 219 of the catheter tube 210 to define a packaging shape of the catheter 200 in the packaging state. In some embodiments, the packaging shape may include a number (i.e., 1, 2, or more) of loops 251 of catheter tube 210.

FIG. 2B illustrates the catheter 200 in a semi-unpackaged state, i.e., a state where the catheter tube 210 is separated from the attachment mechanism 234. As shown, the attachment mechanism 234 may be configured to facilitate separation by the user applying a pulling force 260 applied to the pull ring 224 and an opposite force 261 applied to the outlet collar 232. The attachment mechanism 234 may include clip as shown or any other suitable mechanism to secure the outlet collar 232 to the medial portion 219 such that the outlet collar 232 (i) remains attached to medial portion 219 in the absence of the pulling force 260 applied to the pull ring 224 and (ii) separates from the medial portion 219 when the pulling force 260 is applied.

FIG. 2C illustrates the catheter 200 in a fully unpackaged state, i.e., a state where the outlet collar 232 is separated from the inlet collar 231. In the packaged and semi-packaged states, the outlet collar 232 is attached to the inlet collar 231 so that outlet collar 232 remains attached to the inlet collar 231 in the absence of a deliberation action by the user, i.e., so that the outlet collar 232 remains attached to the inlet collar 231 until the user is ready to deploy the catheter 200. The outlet collar 232 includes an inlet connection member 226 configured to facilitate coupling of the inlet collar 231 with the outlet collar 232. Similarly, the inlet collar 231 includes a corresponding outlet connection member 225 configured to facilitate coupling of the outlet collar 232 with the inlet collar 231. The inlet and outlet connection members 226, 225 are configured to enable separation of the outlet collar 232 from the inlet collar 231 by applying a pull force 264 to the outlet collar 232 or the outlet section 214 and applying opposite pull force 265 to the inlet collar 231 or inlet section 213.

The catheter 200 may be configured for clean disposal of the catheter 200 after use. As such, the inlet and outlet connection members 226, 225 may facilitate re-attachment of the outlet collar 232 with the inlet collar 231 so that the user may re-attach the outlet collar 232 to the inlet collar 231 by applying opposite pushing forces (forces opposite the pulling forces 264, 265) to the outlet collar 232 and the inlet collar 231. Re-attachment of the outlet collar 232 to the inlet collar 231 may redefine a seal between the outlet collar 232 and the inlet collar 231 thereby redefining the closed space 216 to contain any residual urine within the lumen 215.

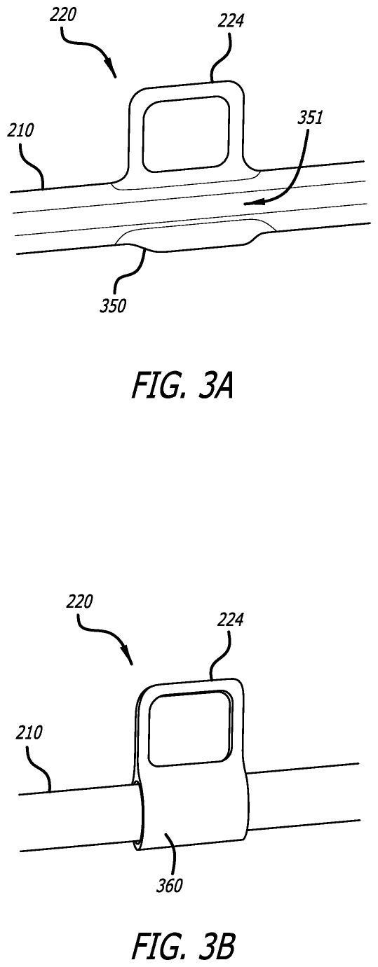

FIG. 3A illustrates a first embodiment of the handling member 220 including an attachment mechanism 350 for securing the pull ring 224 to the catheter tube 210. The attachment mechanism 350 may extend partially around the catheter tube 210 so as to define a gap 351. In some embodiments, the attachment mechanism 350 may extend more than 180 degrees around the catheter tube 210 to grip or partially encapsulate the catheter tube 210. In some embodiments, the attachment mechanism 350 may include an adhesive bond. In other embodiments, the attachment mechanism 350 may include a thermal bond (a bond defined by heat) such as heat staking, or over molding.

FIG. 3B illustrates a second embodiment of the handling member 220 including the attachment mechanism 360 for securing the pull ring 224 to the catheter tube 210. The attachment mechanism 360 may extend entirely around the catheter tube 210 so as to fully encapsulate the catheter tube 210. In some embodiments, the attachment mechanism 350 may define an interference fit with the catheter tube 210 to inhibit sliding displacement of the pull ring 224 along the catheter tube 210. In other embodiments, the attachment mechanism 350 may include a thermal bond (a bond defined by heat) such as heat staking, or over molding.

Methods of using the catheter to drain urine from the bladder may include all or any subset of the following steps or process as self-performed by the user. Methods described herein may refer to male anatomy. However, the methods are not limited to male users, and as such, the methods disclosed herein may also be performed by female users. In some embodiments, the catheter may be enclosed within an outer package such as a bag or pouch, for example. As such, a step may include the user removing the catheter from the outer package.

Another step may include the user breaching the closed volume of the catheter. Such a breaching step may include separating each of the distal and proximal ends of the catheter tube from their corresponding coupling members. In some embodiments, separating each of the distal and a proximal ends may include decoupling the distal and a proximal ends from a handling member of the catheter, where the handling member is coupled with the catheter tube. In other embodiments, separating each of the distal and a proximal ends may include decoupling the distal and a proximal ends from each other.

Another step may include draining the lubricant from the lumen of the catheter tube. Draining the lubricant may include transitioning the catheter tube from a looped shape toward an extended shape. Draining the lubricant may also include orienting the catheter tube toward a vertical attitude so that gravity may induce the lubricant to drain from lumen toward the proximal end or the distal end.

Another step may include displacing the inlet collar along the inlet section away from the distal end to expose the distal tip of the inlet section. In some embodiments, displacing the inlet collar along the inlet section may include positioning the inlet collar at a first location that enables the user to define an orientation and/or position of the distal tip by grasping the inlet collar.

Another step may include orienting and/or positioning the distal end of the catheter tube in relation the urethra of the user. In some embodiments, orienting and/or positioning the distal end may include grasping and manipulating the orientation/position of the inlet collar to orient and/or position the distal tip.

Another step may include inserting the distal tip of the inlet section into the urethra. In some embodiments, inserting the distal tip may include grasping the inlet collar with one hand and grasping the handling member with the other hand. In some embodiments, inserting the distal tip may further include maintaining a position the inlet collar adjacent the urethra and applying an axial force to the handling member to cause the distal tip to enter the urethra.

Another step may include advancing the inlet section along the urethra to the bladder. In some embodiments, advancing the inlet section may include maintaining a position the inlet collar adjacent the urethra and applying a pushing axial force to the handling member to cause displacement of the inlet section along the urethra. In some embodiments, advancing the inlet section may include sliding the inlet section through the inlet collar. In some embodiments, advancing the inlet section may further include positioning the inlet collar at a second location along the inlet section, where the section location is proximal the first location. In some embodiments, the second location may be adjacent the handling member.

Another step may include draining the urine from the bladder. In some embodiments, draining the urine from the bladder may include placing the proximal end of the tube at a lower vertical position than the distal end to cause the urine to flow: (i) into the openings, (ii) proximally along the lumen, and (iii) out of the proximal end. In some embodiments, may draining the urine from the bladder include positioning the proximal end of the tube over a urine receptacle, such as a toilet, for example to cause the urine to enter the receptacle.

Another step may include withdrawing the inlet section from the urethra. In some embodiments, withdrawing the inlet section may include maintaining a position of the inlet collar adjacent the urethra and applying a pulling axial force to the handling member to cause withdrawal of the inlet section from the urethra. In some embodiments, withdrawing the inlet section may include sliding the inlet section through the inlet collar. In some embodiments, withdrawing the inlet section may further include positioning the inlet collar at the distal end of the inlet section.

Another step may include re-establishing the closed volume to prevent any residual urine within the lumen from exiting the lumen. In some embodiments, re-establishing the closed volume may include recoupling the distal and proximal ends to the handling member or to each other. In some embodiments, re-establishing the closed volume may include transitioning the catheter from the extended shape to the looped shape.

Another step may include disposing the catheter in a trash receptacle. In some embodiments, disposing the catheter may include replacing the catheter into the outer package.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A urinary catheter comprising:
a catheter tube defining a distal end and a proximal end, the catheter tube including:
an inlet section extending proximally away from the distal end, the inlet section configured for advancement within a urethra of a user,
an outlet section extending proximally away from the inlet section, and
a lumen extending between the proximal end and the distal end;
wherein the proximal end is coupled with an outlet connection member of the catheter to define a loop of the catheter tube.

2. The catheter of claim 1, wherein the distal end is coupled with an inlet connection member of the catheter to define a closed volume extending along the lumen from the distal end to the proximal end.

3. The catheter of claim 2, wherein:
the distal end is configured to selectively couple with and decouple from the inlet connection member, and
the proximal end is configured to selectively couple with and decouple from the outlet connection member.

4. The catheter of claim 2, further comprising:
a lubricant contained within the closed volume.

5. The catheter of claim 1, further comprising:
an inlet collar coupled with the catheter tube, the inlet collar positionable along the inlet section between the distal end of the catheter tube and a proximal end of the inlet section.

6. The catheter of claim 5, wherein the inlet collar includes the outlet connection member.

7. The catheter of claim 5, further comprising a handling member attached to the catheter tube along the outlet section, the handling member configured for grasping by a user during deployment of the catheter.

8. The catheter of claim 7, wherein:
the handling member includes grasping features disposed along an exterior surface of the handling member to enhance grasping of handling member by the user, and
the grasping features include one or more of protrusions, depressions, axial ribs, axial troughs, annular ribs, annular troughs, or a textured surface.

9. The catheter of claim 7, wherein the handling member includes the outlet connection member and the inlet connection member so that:
the outlet section defines a loop, and
the inlet section defines a loop.

10. The catheter of claim 9, wherein:
the proximal end is configured to decouple from the outlet connection member in response to an axial separation force applied to the outlet section, and
the distal end is configured to decouple from the inlet connection member in response to an axial separation force applied to the inlet section.

11. The catheter of claim 9, wherein the distal end is coupled with the inlet connection member via the inlet collar.

12. The catheter of claim 9, wherein the outlet connection member and the inlet connection member are arranged to define a parallel orientation of the proximal and distal ends of the catheter tube.

13. The catheter of claim 9, wherein the outlet connection member and the inlet connection member are arranged side by side to enable simultaneous application of the axial forces to the outlet section and the inlet section.

14. The catheter of claim 9, further comprising an outlet collar fixedly attached to catheter tube adjacent the proximal end, wherein the proximal end of the catheter tube is coupled with the outlet connection member via the outlet collar.

15. The catheter of claim 14, wherein the inlet collar and/or the outlet collar include one or more of the grasping features disposed along an outside surface.

16. The catheter of claim 5, wherein:
the inlet section includes a number of openings adjacent the distal end, the openings extending between the lumen and an outside surface of the inlet section, and
the inlet collar is configured to cover the openings when the inlet collar is positioned at the distal end.

17. A urinary catheter, comprising:
a catheter tube configured for insertion within a urethra, the catheter tube defining a lumen extending between a proximal end and a distal end,
wherein the catheter is transitionable between a packaging state and a ready-to-use state such that:
in the packaging state, a lubricant is sealably contained within the lumen between the proximal end and the distal end, and
in the ready-to-use state, the lubricant is drained from the lumen.

18. The catheter of claim 17, wherein in the packaging state, the catheter tube defines a shape having two or more loops.

19. The catheter of claim 17, wherein in the packaging state, the proximal end is positioned adjacent the distal end.

20. The catheter of claim 17, wherein in the packaging state, the proximal end and the distal end are oriented parallel with each other.

21. The catheter of claim 17, wherein in the packaging state, the proximal end and the distal end face the same direction.

22-30. (canceled).
